(19) United States
(12) Patent Application Publication
(10) Pub. No.: US 2004/0057935 A1
(43) Pub. Date: Mar. 25, 2004

(54) Intratumoral delivery of dendritic cells

(75) Inventors: John Yu, Los Angeles, CA (US); Keith Black, Los Angeles, CA (US); Moneeb Ehtesham, Los Angeles, CA (US)

Correspondence Address: Pillsbury Winthrop LLP, Intellectual Property Group, Suite 2800, 725 South Figueroa Street, Los Angeles, CA, 90017-5406, US

(73) Assignee: CEDARS-SINAI MEDICAL CENTER

(21) Appl. No.: 10/251,148

(22) Filed: Sep. 20, 2002

(51) Int. Cl. A61K 45/00

(52) U.S. Cl. 424/93.7

(57) ABSTRACT

Methods included herein describe the treatment of a tumor by administering dendritic cells either directly into the same or into its surrounding tissue. Further methods describe the induction of immune cell infiltration into tumors and the treatment of tumors with unprimed dendritic cells by administering dendritic cells in a similar fashion. The methods of the present invention are particularly advantageous in the treatment of brain tumors and other solid tumors disposed throughout the body of a mammal that are difficult or impossible to treat by conventional surgical means. Dendritic cell-based compositions effective in the treatment of such tumors are also described.

### FIELD OF THE INVENTION

The present invention relates to methods of treating a tumor by administering dendritic cells and compositions effective for the same. More specifically, the method involves administering dendritic cells directly into a tumor or its surrounding tissue, the tumor being located in the body of a mammal. The compositions are dendritic cell-based.

### BACKGROUND OF THE INVENTION

Cancer remains one of the leading causes of death in the United States and around the world. Various forms of cancer are differentially treated, depending in part on the location of a tumor targeted for treatment. One particularly difficult group of tumors to treat are those that reside in and near the brain. Treatment of brain tumors presents a number of problems, not the least of which being the dangers inherent in any surgical procedure involving regions of the brain and the tissue located nearby. There is little room for error and the consequences of even a minor surgical mishap can be devastating to a patient; brain damage, or even death may result. Still, where possible, surgery remains the preferred method of treatment for most brain tumors and is often performed in conjunction with radiation therapy and chemotherapy. However, even commonly referenced medical authority suggests that patients with brain tumors be referred to centers specializing in investigative therapies; an indication that conventional modes of treatment are not overwhelmingly successful.

Glioblastoma multiforme and anaplastic astrocytomas are classified in the category of brain tumors commonly known as malignant gliomas. Although not particularly common tumors themselves, they represent a class of tumors associated with significant rates of mortality and morbidity. Current treatment for malignant glioma consists of surgical resection followed by radiation therapy and chemotherapy. However, this treatment generally fails in substantially changing the outcome for a patient; median survival remains less than one year even with medical intervention.

Inducing the body's immune system to specifically combat tumor cells may ultimately be the only means of completely eliminating these cells. Such an immunotherapy approach was attempted over a decade ago, by treating cancer patients with a combination of recombinant human interleukin-2 (IL-2) and lymphokine-activated killer (LAK) cells. Although this form of immunotherapy was well-tolerated by patients, therapeutic attempts implementing the same have thus far not led to the identification of a superior treatment or cure.

More recent studies have focused on other means of initiating and promulgating an immune response by activating T-cells and targeting the same against infiltrative tumor cells. Generally, the body's immune response is initiated when antigen presenting cells (APCs) digest an antigen into fragments, and subsequently present this digested antigen to T-cells. T-cells recognize the digested fragments and bind to the APCs; this activates the T-cells and triggers the immune response.

Some studies suggest that tumor cells themselves may contain immunogeneic antigens. However, these studies further note that tumor cells are poor APCs; they do not efficiently internalize and process/present tumor antigens to T-cells. S. Constant et al., “Peptide and protein antigens require distinct antigen-presenting cell subsets for the priming of CD4+ T cells,” J. Immunol. 154:4915-4923 (1995); D. Levin et al., “Role of dendritic cells in the priming of CD4+ lymphocytes to peptide antigen in vivo,” J. Immunol. 151:6742-6748 (1993). Thus, additional support and/or stimulation is required to trigger a more substantial and effective immune response.

To aid in the stimulation of such an immune response and to increase tumor cell immunogenicity, vaccination with genetically engineered cells expressing a variety of cytokines has been attempted. Cytokines are known to induce immune and inflammatory responses, and various studies have shown these responses to exhibit an anti-tumor effect. G. Dranoff et al., “Vaccination with irradiated tumor cells engineered to secrete murine granulocyte-macrophage colony-stimulating factor stimulates potent, specific, and long-lasting anti-tumor immunity,” Proc. Natl. Acad. Sci. USA 90:3539-3543 (1993). Moreover, it is believed that this anti-tumor effect is initiated by the cytokines' role in antigen presentation through recruitment of APCs such as macrophages and B-cells, which can, in turn, activate primed T-cells. For instance, treatment modalities involving interleukin-4 (IL-4), transforming growth factor-β(TGF-β), and granulocyte-macrophage colony-stimulating factor (GM-CSF) have each been successfully examined for an anti-tumor effect. In fact, GM-CSF has been identified as the most potent cytokine for achieving brain tumor-specific immunity, when cells expressing this cytokine are vaccinated peripherally. Dranoff et al. at 3539. Although the isolation of a ubiquitous tumor antigen has been elusive, by vaccinating peripherally with cells expressing various cytokines, the body's immune response may be triggered and targeted against an intracranial tumor. However, intracranial vaccination with cytokine-expressing cells is not clinically implemented, as intracranial cytokine expression presents a host of potential complications, such as the undesirable induction of an inflammatory response within the brain that is not targeted against tumor cells.

Although B-cells, macrophages, and other APCs recruited by cytokines may aid in the activation of an immune response, the most potent APCs in the body are dendritic cells. Dendritic cells are “professional APCs,” as they are uniquely capable of activating both primed T-cells (as in the case of macrophages and B-cells) and naive T-cells (i.e., those that have not previously encountered antigens). In fact, dendritic cells are the only APCs known to process exogenous antigen through the class I pathway. Thus, efforts have been directed toward determining whether dendritic cells presenting tumor-associated antigens can mediate a significant anti-tumor response; potentially an anti-tumor response stronger than that induced by cytokine-presenting cells.

In murine models and in two published clinical trials, cytokine-stimulated dendritic cells have been pulsed ex vivo with tumor antigens and used successfully as anti-tumor vaccines for extracranial tumor models. J. Mayorodomo et al., “Bone marrow-derived dendritic cells pulsed with synthetic tumor peptides elicit protective and therapeutic antitumor immunity,” Nature Med. 1:1297-1302 (1995); J. Young and K. Inaba, “Dendritic cells and adjuvants for class I major histocompatability complex-restricted antitumor immunity,” J. Exp. Med. 183:7-11 (1996); L. Zitvogel et al., “Therapy of murine tumors with tumor peptide-pulsed dendritic cells: dependence on T cells, B7 costimulation, and T helper cell 1-associated cytokines,” J. Exp. Med. 183:87-97 (1996); F. Hsu et al., “Vaccination of patients with B-cell lymphoma using autologous antigen-pulsed dendritic cells,” Nature Med. 2:52-58 (1996); and F. O. Nestle et al., “Vaccination of melanoma patients with peptide- or tumor-lysate pulsed dendritic cells,” Nature Med. 4:328-332 (1998). In addition, another study demonstrated the use of tumor peptide-pulsed APCs to successfully treat a murine model of a metastatic intracranial tumor model. D. M. Ashley et al., “Bone marrow-generated dendritic cells pulsed with tumor extracts or tumor RNA induce antitumor immunity against central nervous systems tumors,” J. Exp. Med. 186:1177-1182 (1997). Moreover, dendritic cell vaccination in human brain tumor patients has demonstrated encouraging results. J. S. Yu et al., “Vaccination of Malignant Glioma Patients with Peptide-pulsed Dendritic Cells Elicits Systemic Cytotoxicity and Intracranial T-cell Infiltration,” Cancer Res. 61:842-847 (2001).

The key limitation in each of these dendritic cell-based vaccination strategies is their reliance on the acquisition of tumor tissue as a protein source for use in priming dendritic cells ex vivo. Priming generally involves culturing the dendritic cells with the tumor cells against which they will subsequently be utilized. This process provides the dendritic cells access to the tumor proteins, thereby allowing the cells to process the associated antigens in preparation for presentation of the digested antigens to T-cells upon administration to a patient. However, priming the dendritic cells in this fashion precludes the use of this therapeutic modality in cases where tumor tissue cannot be readily obtained; a shortcoming frequently encountered with various types of tumors, since a variety of circumstances can render ex vivo priming either impractical or impossible. For instance, a tumor may be surgically inaccessible, or the surgical manipulation thereof may present unreasonable danger to the health and safety of a patient. Even in instances where tumor tissue can be readily accessed and sampled, the surgical harvest required to obtain that tissue subjects a patient to yet another procedure, the avoidance of which is likely desirable.

There is therefore a need in the art for a system and method of implementing a dendritic cell-based vaccination strategy that obviates, for practical purposes, the above-mentioned limitations. More specifically, there is a need in the art for a composition including and method for implementing a dendritic cell-based vaccination strategy to treat a tumor without the need for ex vivo priming of the dendritic cells prior to administration of the same to a patient.

### SUMMARY OF THE DISCLOSURE

The present invention provides a method and dendritic cell-based composition for treating a tumor. The method may include administering dendritic cells directly into the tumor itself or into the tissue surrounding or located nearby the tumor, and may be effective in the treatment of a tumor disposed in any location throughout the body of a mammal. The method and composition do not require the dendritic cells used therein to be primed ex vivo prior to inclusion in the composition or administration to a patient. The methods of the present invention may induce immune cell infiltration into a tumor.

Other features and advantages of the invention will become apparent from the following detailed description, which illustrates, by way of example, various embodiments of the present invention. Certain embodiments may be especially advantageous in the treatment of brain tumors and other solid tumors that are difficult to treat by various conventional means.

### BRIEF DESCRIPTION OF THE FIGURES

The file of this patent contains at least one drawing executed in color. Copies of this patent with color drawing(s) will be provided by the patent and Trademark Office upon request and payment of the necessary fee.

FIG. 1 depicts a phenotypic profile of dendritic cells in accordance with an embodiment of the present invention. Bone marrow cultures yielded cells expressing cell surface phenotypic markers that dendritic cells commonly express.

FIG. 2 depicts an inhibition of tumor growth owing to intratumoral dendritic cell vaccination in accordance with an embodiment of the present invention. Dendritic cells (Column B) and a saline control (Column A) were vaccinated into subcutaneous 9L glioma tumors on the dorsum of the foot. Tumor volume was markedly lower when treated with dendritic cells.

FIG. 3 depicts an intratumoral vaccination with dendritic cells inducing T-cell infiltration into brain tumors in accordance with an embodiment of the present invention. Dendritic cells vaccinated into brain tumors induce increased CD4+ T-cell infiltration (FIG. 3A) as compared to saline-treated controls (FIG. 3B).

FIG. 4 depicts an intracranial dendritic cell vaccination prolonging survival in accordance with an embodiment of the present invention. Fisher rats with 9L LacZ brain tumors when treated with intracranial dendritic cell vaccination survived longer than saline-inoculated controls, with 73% (n=18) of the dendritic cell treated animals surviving past 90 days as compared to 8% (n=12) of the controls.

FIG. 5 depicts an intracranial dendritic cell vaccination prolonging survival in animals with established intracranial gliomas in accordance with an embodiment of the present invention. When vaccinated intracranially with dendritic cells, 9L glioma-bearing rats survived longer than monocyte-inoculated controls.

FIG. 6 depicts a promotion of T-cell infiltration in 9L intracranial gliomas inoculated with dendritic cells in accordance with an embodiment of the present invention. Strong T-cell infiltration was observed in animals vaccinated with dendritic cells (FIG. 6A) and weak infiltration was observed in monocyte-treated (FIG. 6B) and saline-treated (FIG. 6C) controls. R2 indicates region of CD8+ staining and R3 indicates region of CD4+ staining.

FIG. 7 is executed in color and depicts a migration of dendritic cells into systemic lymph nodes in accordance with an embodiment of the present invention. Dendritic cells expressing green fluorescent protein (GFP) were found dispersed within the main tumor mass (FIG. 7A) and within deep cervical lymph nodes ipsilateral to the site of implantation (FIG. 7B); however no appreciable GFP positivity was indicated in contralateral cervical lymph node tissue (not shown) or in lymph nodes from animals not inoculated with dendritic cells (FIG. 7C).

FIG. 8 depicts an enhancement of tumor-specific cytotoxic T-cell activity by intratumoral inoculation of dendritic cells into intracranial brain tumors in accordance with an embodiment of the present invention. T-cells from animals inoculated with dendritic cells demonstrated a 1.48-fold increase in IFN-gamma RNA message compared to monocyte-treated (1.12-fold) and saline-treated (1.20-fold) controls.

FIG. 9 depicts an enhancement of tumor-specific cytotoxic T-cell activity by intratumoral inoculation of dendritic cells into intracranial brain tumors in accordance with an embodiment of the present invention. T-cells from animals inoculated with dendritic cells demonstrated a 1.31-fold increase in secreted IFN-gamma compared to monocyte-treated (0.70-fold) and saline-treated (1.10-fold) controls.

### DETAILED DESCRIPTION OF THE INVENTION

The present invention is based upon a composition for and method of treating a tumor by injecting or otherwise administering dendritic cells into the tumor or its immediately surrounding tissue (hereinafter, a tumor and its immediately surrounding tissue are collectively included in the term “tumor region”). More specifically, the method involves injecting or otherwise administering the dendritic cells directly into a solid tumor. Treating a tumor in accordance with the embodiments of the present invention provides dendritic cells with more direct access to the tumor than that which is possible with conventional methods; especially those conventional methods wherein dendritic cells are administered peripherally. The techniques of the present invention may be particularly advantageous in instances where a tumor is surgically inoperable, where surgery is otherwise undesirable, or where no portion of the tumor can be retrieved for priming dendritic cells ex vivo against the tumor; although such factors need not be indicated in order for the methods of the present invention to be effective.

Owing in part to the difficulty in accessing and treating brain tumors with conventional surgical methods, the methods of the present invention may be especially advantageous in the treatment of tumors located in the brain of a mammal; particularly in treating high- or low-grade malignant gliomas, and, even more particularly, in treating anaplastic astrocytoma or glioblastoma multiforme. However, it should be recognized that numerous other types of tumors, and especially solid tumors, located in a variety of locations throughout the body of a patient may be treated in accordance with the methods of the present invention. Such other locations may include, but are in no way limited to, the skin (e.g., melanomas), breast, gastrointestinal tract, or respiratory tract.

The composition and methods of the present invention are based, in part, on the inventors' surprising discovery that unprimed dendritic cells may be delivered to the tumor region of a patient and may thereafter be effective in the treatment of the tumor. Dendritic cells administered in this manner essentially prime themselves in vivo upon coming into contact or otherwise establishing biochemical communication with the target tumor cells, and, correspondingly, their antigen proteins.

Unprimed dendritic cells include those dendritic cells that do not rely upon the acquisition of tumor tissue as a protein source, and the subsequent culturing therewith. In conventional methods, as discussed above, dendritic cells are primed ex vivo. This generally involves culturing the dendritic cells with the tumor cells against which they will subsequently be utilized. This process provides the dendritic cells access to the tumor proteins, thereby allowing the cells to process the associated tumor antigens and prepare to present the digested antigens to T-cells upon introduction of the dendritic cells to the body of a patient. However, in various embodiments of the present invention, dendritic cells may be delivered directly into a tumor bed or tumor region without first being primed ex vivo; the dendritic cells process the tumor antigens only in vivo.

Dendritic cells suitable for use in accordance with the present invention may be isolated or obtained from any tissue in which such cells are found, or may be otherwise cultured and provided. In particular, antigen-presenting dendritic cells are preferred for use in accordance with the methods of the present invention. Such dendritic cells may be found, by way of example, in the bone marrow or peripheral blood mononuclear cells (PBMCs) of a mammal, in the spleen of a mammal, or in the skin of a mammal (i.e., Langerhan's cells, which possess certain qualities similar to that of dendritic cells, may be found in the skin and may further be employed in conjunction with the methods of the present invention, and are included within the scope of the term “dendritic cells” as used herein). In the most preferred embodiments of the present invention, cells obtained from the bone marrow of a mammal may be utilized. Therefore, in one embodiment of the present invention, bone marrow may be harvested from a mammal and cultured in a medium. Any suitable medium that promotes the growth of dendritic cells may be used in accordance with the present invention, and may be readily ascertained by one of skill in the art without undue experimentation.

In one embodiment of the present invention, GM-CSF and/or IL-4 may be included in the above-described medium. Media may be at least partially replenished every few (e.g., two to four) days during the culturing process. After a suitable amount of time, clusters of dendritic cells may be apparent in the medium, and may be retrieved therefrom, either in individual clusters or in any other convenient amount. Quantities of dendritic cells may be subcultured, where desirable, to generate yet greater quantities of the same.

Dendritic cells used in conjunction with the methods of the present invention may be delivered to a tumor region (e.g., a brain tumor, or the surrounding brain tissue) in a recipient by any suitable means. Such means of delivery may include, but are in no way limited to, injection, infusion, inoculation, direct surgical delivery, or any combination thereof. In a preferred embodiment of the present invention, dendritic cells may be administered to a mammal by direct inoculation via stereotactic surgery; a standard inoculation procedure known to those of skill in the art of neurosurgery. Further appropriate mechanisms for delivering dendritic cells to a tumor or its surrounding tissue will be readily apparent, to one in the art without undue experimentation, and are contemplated as being within the scope of the present invention.

The composition of the present invention may include unprimed dendritic cells in a pharmaceutical carrier. Any conventional pharmaceutical carrier may be used in accordance with the composition or methods of the present invention, and an appropriate carrier may be selected by one of skill in the art without undue experimentation. In one embodiment of the present invention, the pharmaceutical carrier is saline, although other carriers may be utilized depending upon the desired characteristics of the composition. For example, one may formulate a composition differently in order to account for different delivery techniques for the composition, physiological differences among patients (e.g., sex, weight, age, etc.), or different types of tumors (e.g., brain, breast, lung, etc.), among other factors.

The dendritic cells administered to a patient in accordance with the composition and methods of the present invention may be delivered in combination with any of a variety of additional substances and compounds. By way of example, the dendritic cells of the present invention may be administered to a patient along with any suitable carrier, vehicle, additive, excipient, pharmaceutical adjunct, or other suitable product, as will be readily ascertained and appreciated by one of skill in the art. Moreover, the dendritic cells of the present invention may be administered in conjunction with other therapeutic compounds or agents useful in the treatment of the tumor or the relief of pain associated with the tumor or treatment thereof.

The quantity of dendritic cells appropriate for administration to a patient to effect the methods of the present invention and the most convenient route of such administration may be based upon a variety of factors, as may the formulation of the composition of the present invention. Some of these factors may include, but are in no way limited to, the physical characteristics of the patient (e.g., age, weight, sex, etc.), the physical characteristics of the tumor (e.g., location, size, rate of growth, accessibility, etc.), and the extent to which other therapeutic means are being simultaneously implemented along with the methods of the present invention (e.g., chemotherapy, beam radiation therapy, etc.). Notwithstanding the variety of factors one should consider in implementing the methods of the present invention to treat a tumor, in a preferred embodiment of the present invention, a patient may be administered with from about 10⁵ to about 10⁷ dendritic cells in from about 0.05 mL to about 0.30 mL saline in a single administration. Additional administrations may be necessary, depending upon the above-described and other factors, such as the severity of tumor pathology. In preferred embodiments of the present invention, from about one to about five administrations of 10⁶ dendritic cells is performed at two-week intervals.

In a preferred embodiment of the present invention, a mammal is treated with dendritic cells following or in conjunction with radiotherapy. While not wishing to be bound by any theory, it is believed that prior or simultaneous treatment with radiotherapy renders a dendritic cell vaccination more effective as is allows the dendritic cells to better process dying tumor cells. Similarly, a chemotherapy regimen administered either prior to or simultaneous with dendritic cell vaccination therapy that induces tumor cells to undergo apoptosis (i.e., programmed cell death) may be beneficial.

As used herein, “treating” a tumor includes, but is not limited to, ameliorating the tumor, lessening the severity of its complications, causing it to decrease in mass and/or size, preventing it from manifesting, preventing it from recurring, merely preventing it from worsening, or a therapeutic effort to effect any of the aforementioned, even if such therapeutic effort is ultimately unsuccessful.

### EXAMPLES

The Examples discussed herein demonstrate that dendritic cells may inhibit the growth of brain tumors when implanted directly into the tumors, and that this treatment may prolong the life of a patient with a brain tumor. The Examples further illustrate that dendritic cell vaccination may induce immune cell infiltration into brain tumors and systemic lymph nodes. Moreover, the Examples show that the implantation of dendritic cells directly into tumors disposed in locations throughout the body of a mammal is effective in the treatment of the same.

### Example 1

### Intracranial Dendritic Cell Vaccination of Brain Tumors

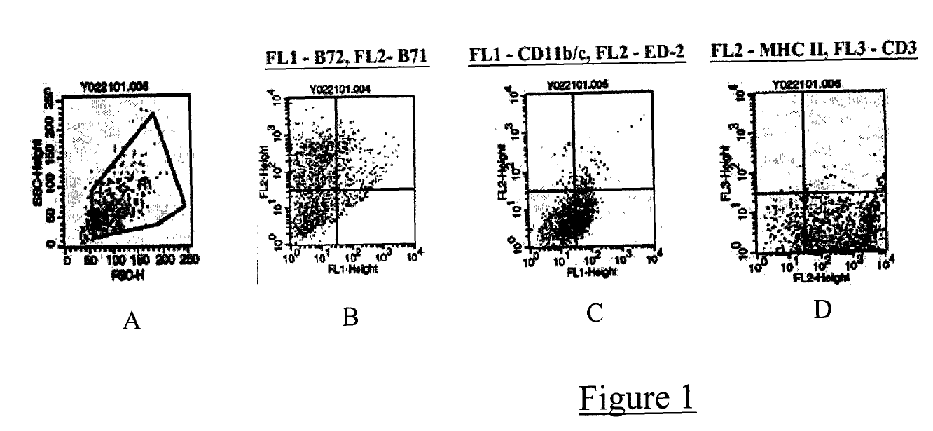

Bone marrow was harvested from the femurs and tibias of adult Fisher rats. Cells were plated in 24 well plates at a density of 1 million cells per well in RMPI 1640 medium (obtained from Gibco BRL; Gaithersburg, Md.; hereinafter “Gibco”) in media containing GM-CSF and IL-4 (both available from R and D Systems; Minneapolis, Minn.; hereinafter “R and D”). Media was partially replenished every three days. After eight days, clusters of enlarged floating/partially adherent dendritic cells were apparent. These cells were collected separately and their phenotypic profiles assessed using flow immunocytometry. They were positive for MHC class II and B7 co-stimulatory molecules; thereby confirming that the cells were dendritic in nature (FIG. 1).

### Example 2

### Dendritic Cells Inhibit Tumor Growth when Inoculated Intratumorally

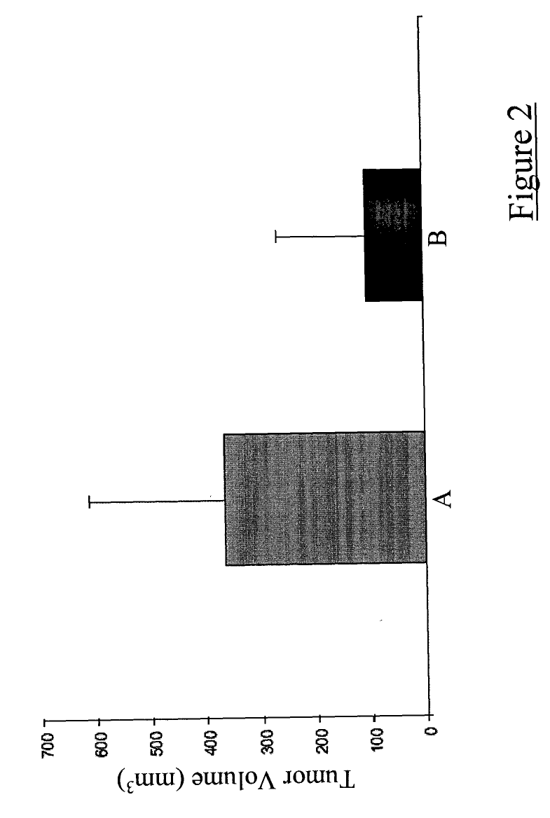

Dendritic cells were inoculated subcutaneously along with a mixture of irradiated and viable 9L glioma cells into the dorsum of the right foot of adult Fisher rats. Two weeks following this procedure, a second dose of dendritic cells was inoculated into each growing tumor. Eight weeks following the second dendritic cell vaccination, tumor sizes were measured using a precision caliper. Tumors were markedly smaller in animals that had received intratumoral dendritic cell vaccinations as compared to the control animals that received only saline inoculations (FIG. 2).

### Example 3

### Dendritic Cell Vaccination Induces Immune Cell (T-cell) Infiltration into Brain Tumors

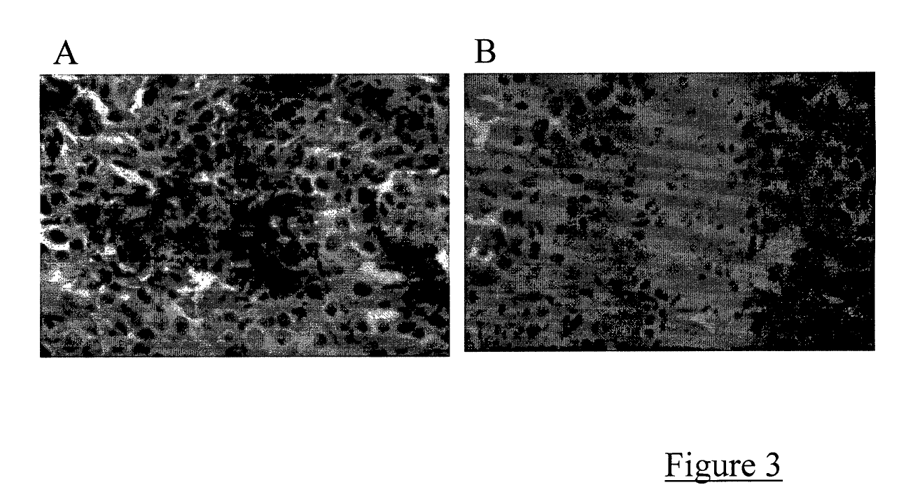

Dendritic cells were inoculated intracranially along with a mixture of irradiated and viable 9L glioma cells into the right corpus striatum (basal ganglia) of adult Fisher rats. Two weeks following this procedure, a second dose of dendritic cells was inoculated into each growing tumor. Two weeks following the second dendritic cell vaccination, animals were euthanized and their brains harvested. The brains were immediately frozen and sectioned on a cryostat (available from Janis Research Company, Inc.; Wilmington, Mass.). Slide mounted sections were stained for T-cell markers (i.e., CD4 and CD8). Tumors from dendritic cell vaccinated animals displayed increased quantities of infiltrating T-cells as compared to tumors from control animals that received only saline inoculations (FIG. 3).

### Example 4

### Dendritic Cell Vaccinations Prolong Survival in Brain Tumor Bearing Rats

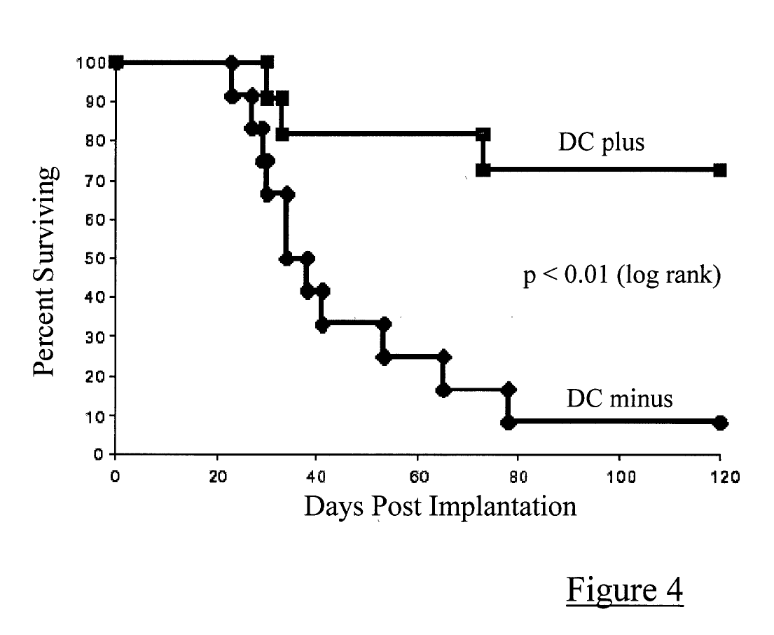

Dendritic cells were inoculated intracranially along with a mixture of irradiated and viable 9L glioma cells into the right corpus striatum (basal ganglia) of adult Fisher rats. Two weeks following this procedure, a second dose of dendritic cells was inoculated into each growing tumor. Control animals were treated at similar time points with intracranial saline inoculations. Animals were followed for survival. Rats treated with intracranial dendritic cell vaccination survived longer than saline treated controls, with 75% of dendritic cell-treated animals surviving beyond 90 days after the initial tumor implantation compared to 5% of the control group (FIG. 4).

### Example 5

### Dendritic Cell Vaccinations Prolong Survival in Rats with Established Brain Tumors

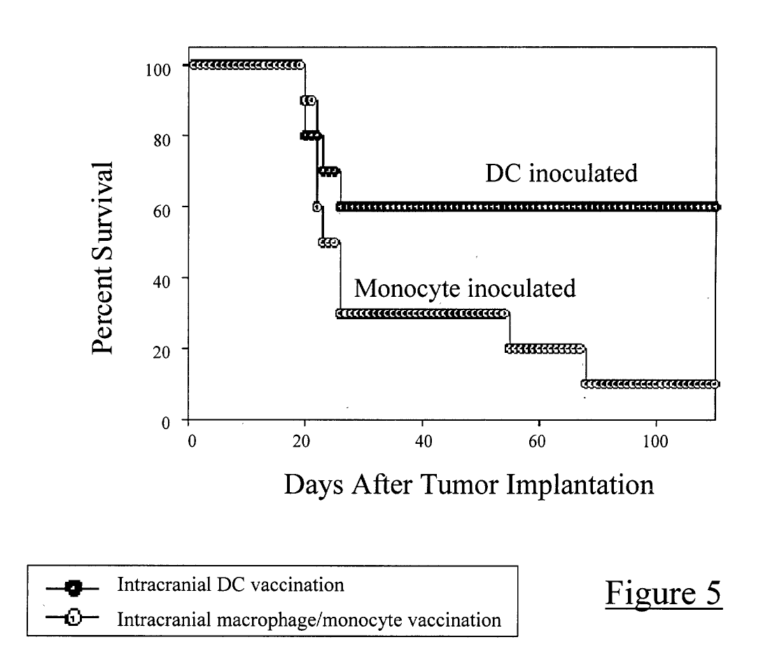

A mixture of irradiated and viable 9L glioma cells was introduced into the right corpus striatum (basal ganglia) of adult Fisher rats. Two days later, rats were inoculated intracranially with dendritic cells. Control animals were treated at similar time points with intracranial monocyte/macrophage inoculations. Animals were followed for survival. Rats treated with intracranial dendritic cell vaccination survived longer than monocyte/macrophage-treated controls, with 60% of dendritic cell-treated animals surviving beyond 90 days after the initial tumor implantation compared to 10% of the control group (FIG. 5). Moreover, surviving rats were immune to intracranial tumor re-challenge.

### Example 6

### Dendritic Cell Vaccinations Promote T-cell Infiltration into Brain Tumors

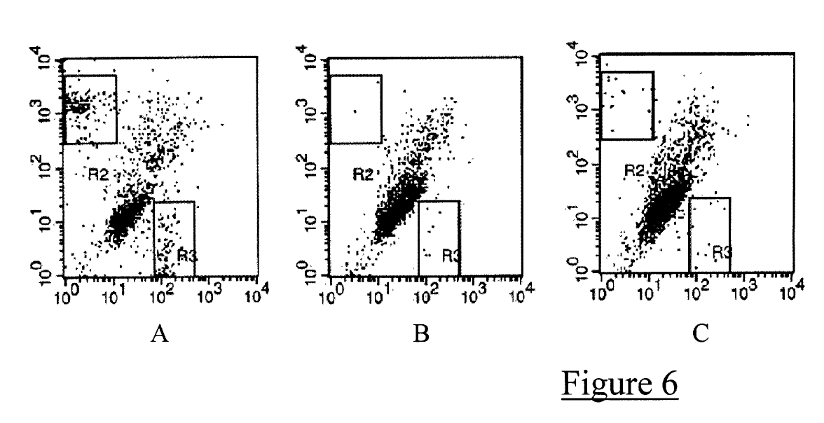

A mixture of irradiated and viable 9L glioma cells was introduced into the right corpus striatum (basal ganglia) of adult Fisher rats. Rats were vaccinated with intratumoral inoculations of immature dendritic cells, monocytes, or saline on days 2 and 16 following tumor implantation. One week following the second intratumoral inoculation, tumors were harvested and stained for CD4+ and CD8+ T-cell content. Results indicated strong T-cell infiltration in animals vaccinated with dendritic cells (FIG. 6A) and weak infiltration in monocyte (FIG. 6B) and saline (FIG. 6C) treated controls.

### Example 7

### Dendritic Cells Migrate to Systemic Lymph Nodes

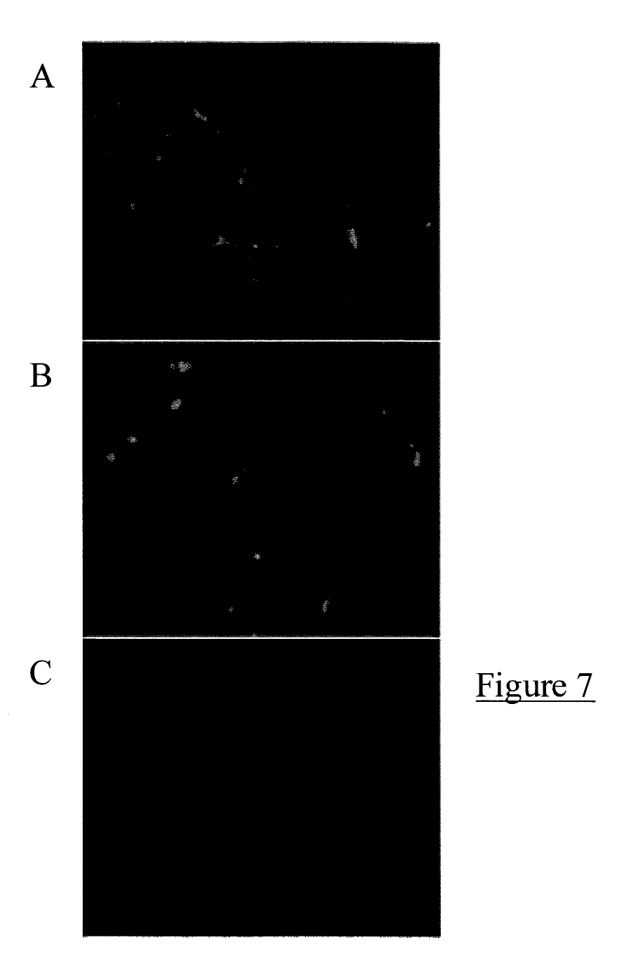

To assess whether dendritic cells inoculated into intracranial brain tumors could drain to the lymphatic system, deep cervical lymph nodes were harvested from rats that had received intracranial co-implantations of partially irradiated 9L glioma cells and green fluorescent protein (GFP) expressing dendritic cells four days earlier. Tumor bearing brain sections from these animals demonstrated GFP positive dendritic cells interspersed within the main tumor mass (FIG. 7A). Deep cervical lymph nodes ipsilateral to the site of implantation were infiltrated with numerous GFP expressing cells (FIG. 7B). In contrast, contralateral cervical lymph node tissue (not shown) or lymph nodes from 9L glioma bearing rats not inoculated with dendritic cells (FIG. 7C) did not reveal any GFP positivity.

### Example 8

### Enhancement of Tumor-Specific Cytotoxic T-cell Activity with Dendritic Cell Inoculation, Measured by Increased IFN-gamma Message

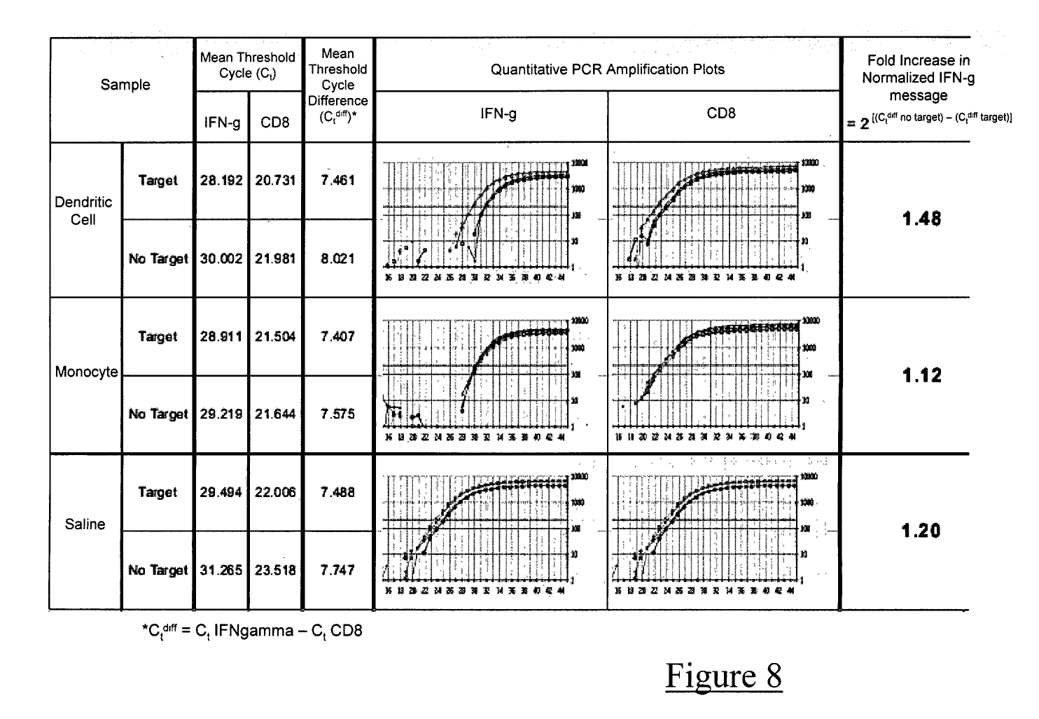

Brain tumor-bearing rats were teated with intratumoral inoculations of dendritic cells, monocytes, or saline (n=4 per group), on days 2 and 16 following tumor implantation. T-cells were isolated from their spleens two weeks following the second intratumoral inoculation. Harvested T-cells were re-stimulated in quadruplicate in vitro with irradiated 9L glioma cells. Two such re-stimulations, each lasting 7 days, were performed on each sample, at the end of which T-cells were either exposed to freshly irradiated 9L glioma cells (FIG. 8, “Target”) or were not re-exposed (FIG. 8, “No Target”).

Four hours following re-exposure, RNA was harvested from T-cells and analyzed by means of a quantitative polymerase chain reaction (PCR) to detect levels of IFN-gamma message.

All samples were also analyzed for CD8 RNA message content, which was used as an internal control, against which IFN-gamma message levels were normalized. Differences in IFN-gamma message levels were compared on the basis of the PCR cycle at which a particular cycle crossed an established threshold. A cycle difference of 1 was assumed to indicate a two-fold difference in the message. Following normalization against CD8 message, a fold-increase in IFN-gamma message for each treatment group was calculated by comparing target with no target.

As depicted in FIG. 8, T-cells from animals inoculated with dendritic cells demonstrated a 1.48-fold increase in IFN-gamma RNA message compared to monocyte (1.12-fold) and saline (1.20-fold) treated controls.

### Example 9

### Enhancement of Tumor-Specific Cytotoxic T-cell Activity with Dendritic Cell Inoculation, Measured by Increased IFN-gamma Secretion

Brain tumor-bearing rats were teated with intratumoral inoculations of dendritic cells, monocytes, or saline (n=4 per group), on days 2 and 16 following tumor implantation. T-cells were isolated from their spleens two weeks following the second intratumoral inoculation. Harvested T-cells were re-stimulated in quadruplicate in vitro with irradiated 9L glioma cells. Two such re-stimulations, each lasting 7 days, were performed on each sample, at the end of which T-cells were either exposed to freshly irradiated 9L glioma cells (FIG. 8, “Target”) or were not re-exposed (FIG. 8, “No Target”).

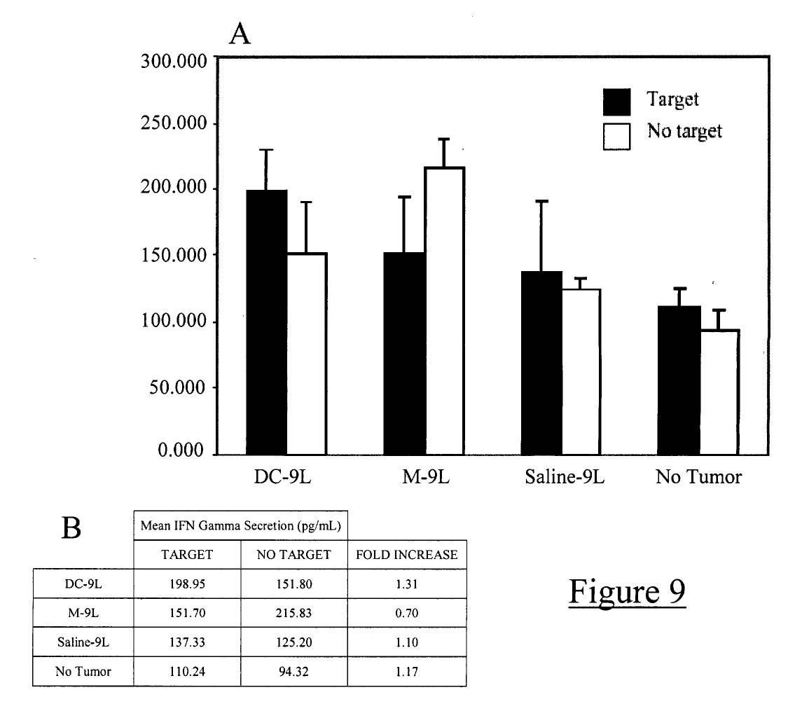

Twenty-four hours following re-exposure, media was harvested from T-cell cultures and analyzed by means of an emzyme-linked immunosorbent assay (ELISA) to quantify IFN-gamma protein secretion. For control purposes, media from re-stimulated T-cells from animals that were not implanted with brain tumors and were therefore never treated (FIG. 9, “No Tumor” group) were also analyzed.

A fold-increase in IFN-gamma levels for each treatment group was calculated by comparing target with no target. As depicted in FIG. 9, T-cells from animals inoculated with dendritic cells demonstrated a 1.31-fold increase in secreted IFN-gamma compared to monocyte (0.70-fold) and saline (1.10-fold) treated controls.

While the description above refers to particular embodiments of the present invention, it will be understood that many modifications may be made without departing from the spirit thereof. For instance, the protease inhibitors of the present invention may be used in the treatment of any number of conditions where inflammation is observed, as would be readily recognized by one skilled in the art and without undue experimentation. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

We claim:

1. A method for treating a tumor in a mammal, comprising:
administering dendritic cells into a region selected from the group consisting of the tumor, tissue surrounding the tumor, and both the tumor and the tissue surrounding the tumor.

2. The method of claim 1, wherein the dendritic cells are unprimed dendritic cells.

3. The method of claim 1, wherein the tumor is a brain tumor and the tissue surrounding the tumor includes at least a portion of the brain of the mammal.

4. The method of claim 1, wherein the tumor is a breast tumor and the tissue surrounding the tumor includes at least a portion of the breast of the mammal.

5. The method of claim 1, wherein the tumor is a gastrointestinal tumor and the tissue surrounding the tumor includes at least a portion of the gastrointestinal tract of the mammal.

6. The method of claim 1, wherein the tumor is a respiratory tumor and the tissue surrounding the tumor includes at least a portion of the respiratory tract of the mammal.

7. The method of claim 1, further comprising harvesting the dendritic cells from a mammal prior to administering the dendritic cells.

8. The method of claim 7, wherein harvesting the dendritic cells from a mammal further includes harvesting the dendritic cells from a source selected from the group consisting of bone marrow of the mammal, peripheral blood mononuclear cells (PMBCs) of the mammal, the spleen of the mammal, and the skin of the mammal.

9. The method of claim 7, further comprising culturing the dendritic cells in a medium after harvesting the dendritic cells.

10. The method of claim 9, wherein the medium comprises granulocyte-macrophage colony-stimulating factor (GM-CSF).

11. The method of claim 9, wherein the medium comprises interleukin-4 (IL-4).

12. The method of claim 9, wherein culturing the dendritic cells in the medium further includes periodically replenishing the medium.

13. The method of claim 9, wherein clusters of dendritic cells form in the medium, and culturing the dendritic cells in the medium further includes collecting the clusters.

14. The method of claim 1, wherein administering the dendritic cells further includes using an administration technique selected from the group consisting of injection, infusion, inoculation, direct surgical delivery, direct inoculation via stereotactic surgery, and a combination thereof.

15. The method of claim 1, wherein administering the dendritic cells further includes administering the dendritic cells in an amount of from about 10⁵ to about 10⁷ dendritic cells in from about 0.05 mL to about 0.30 mL saline.

16. The method of claim 1, wherein administering the dendritic cells further includes performing multiple administrations of the dendritic cells.

17. The method of claim 16, wherein the multiple administrations are performed at an interval of about two weeks.

18. The method of claim 1, wherein the tumor is surgically inoperable.

19. The method of claim 1, wherein the tumor is a solid tumor.

20. The method of claim 1, further comprising administering radiation therapy to the mammal.

21. The method of claim 1, further comprising administering chemotherapy to the mammal.

22. A method for inducing immune cell infiltration into a tumor in a mammal, comprising:
administering dendritic cells into a region selected from the group consisting of the tumor, tissue surrounding the tumor, and both the tumor and the tissue surrounding the tumor.

23. The method of claim 22, wherein the dendritic cells are unprimed dendritic cells.

24. The method of claim 22, wherein the tumor is a brain tumor and the tissue surrounding the tumor includes at least a portion of the brain of the mammal.

25. The method of claim 22, wherein the tumor is a breast tumor and the tissue surrounding the tumor includes at least a portion of the breast of the mammal.

26. The method of claim 22, wherein the tumor is a gastrointestinal tumor and the tissue surrounding the tumor includes at least a portion of the gastrointestinal tract of the mammal.

27. The method of claim 22, wherein the tumor is a respiratory tumor and the tissue surrounding the tumor includes at least a portion of the respiratory tract of the mammal.

28. The method of claim 22, further comprising harvesting the dendritic cells from a mammal prior to administering the dendritic cells.

29. The method of claim 28, wherein harvesting the dendritic cells from a mammal further includes harvesting the dendritic cells from a source selected from the group consisting of bone marrow of the mammal, peripheral blood mononuclear cells (PMBCs) of the mammal, the spleen of the mammal, and the skin of the mammal.

30. The method of claim 28, further comprising culturing the dendritic cells in a medium after harvesting the dendritic cells.

31. The method of claim 30, wherein the medium comprises granulocyte-macrophage colony-stimulating factor (GM-CSF).

32. The method of claim 30, wherein the medium comprises interleukin-4 (IL-4).

33. The method of claim 30, wherein culturing the dendritic cells in the medium further includes periodically replenishing the medium.

34. The method of claim 30, wherein clusters of dendritic cells form in the medium, and culturing the dendritic cells in the medium further includes collecting the clusters.

35. The method of claim 22, wherein administering the dendritic cells further includes using an administration technique selected from the group consisting of injection, infusion, inoculation, direct surgical delivery, direct inoculation via stereotactic surgery, and a combination thereof.

36. The method of claim 22, wherein administering the dendritic cells further includes administering the dendritic cells in an amount of from about 10⁵ to about 10⁷ dendritic cells in from about 0.05 mL to about 0.30 mL saline.

37. The method of claim 22, wherein administering the dendritic cells further includes performing multiple administrations of the dendritic cells.

38. The method of claim 37, wherein the multiple administrations are performed at an interval of about two weeks.

39. The method of claim 22, wherein the tumor is surgically inoperable.

40. The method of claim 22, wherein the tumor is a solid tumor.

41. The method of claim 22, further comprising administering radiation therapy to the mammal.

42. The method of claim 22, further comprising administering chemotherapy to the mammal.

43. A composition for treating a tumor, comprising:
unprimed dendritic cells; and
a pharmaceutical carrier.

44. The composition of claim 43, wherein the unprimed dendritic cells are harvested from a source in a mammal.

45. The composition of claim 44, wherein the source is selected from the group consisting of bone marrow of the mammal, peripheral blood mononuclear cells (PMBCs) of the mammal, the spleen of the mammal, and the skin of the mammal.

46. The composition of claim 43, wherein the pharmaceutical carrier is saline.

47. The composition of claim 46, further comprising from about 10⁵ to about 10⁷ unprimed dendritic cells and from about 0.05 mL to about 0.30 mL saline.

48. The composition of claim 43, wherein the unprimed dendritic cells are grown in a medium.

49. The composition of claim 48, wherein the medium comprises granulocyte-macrophage colony-stimulating factor (GM-CSF).

50. The composition of claim 48, wherein the medium comprises interleukin-4 (IL-4).

51. The composition of claim 43 further comprising an additional component selected from the group consisting of a carrier, a vehicle, an additive, an excipient, a pharmaceutical adjunct, a therapeutic compound or agent useful in the treatment of the tumor, and a therapeutic compound or agent useful in the relief of pain.
